(19) United States
(12) Patent Application Publication
(10) Pub. No.: US 2025/0325006 A1
(43) Pub. Date: Oct. 23, 2025

(54) FOOD INGREDIENT CONVERSION METHOD AND FOOD INGREDIENT CONVERSION DEVICE

(71) Applicant: Panasonic Intellectual Property Management Co., Ltd., Osaka (JP)

(72) Inventors: FUMIYA WAYAMA, Osaka (JP); NORIYUKI HATSUGAI, Kyoto (JP); KIYOTAKA TSUJI, Osaka (JP); YASUAKI OKUMURA, Kyoto (JP); KUNIHIRO UKAI, Nara (JP)

(21) Appl. No.: 19/259,005

(22) Filed: Jul. 3, 2025

Related U.S. Application Data
(63) Continuation of application No. PCT/JP2023/045576, filed on Dec. 19, 2023.

(30) Foreign Application Priority Data
Jan. 26, 2023 (JP) 2023-010235

(51) Int. Cl. A23L 5/20; A23L 15/00

(52) U.S. Cl. CPC A23L 5/25; A23L 15/25

(57) ABSTRACT

A food ingredient conversion method includes activating at least one of an enzyme or a coenzyme by applying a voltage from an external power supply located outside a reaction vessel and connected to a working electrode and a counter electrode to cause a current to flow between the working electrode and the counter electrode, transferring a proton between an organic compound and an external liquid by an enzymatic reaction using at least one of the activated enzyme or coenzyme, acquiring information on the current that has been caused to flow by the external power supply, and determining, based on the acquired information on the current, an amount of a pH adjusting agent to be fed for adjusting a pH of a reaction system.

### BACKGROUND

### 1. Technical Field

The present disclosure relates to a food ingredient conversion method and a food ingredient conversion device.

### 2. Description of the Related Art

In manufacturing of food, converting organic compounds which are ingredients of food is widely performed for the purpose of, for example, modifying food ingredients.

An example of such a food ingredient conversion technique is a technique of using a nickel catalyst in order to hydrogenate fat and oil components serving as raw materials in manufacturing of margarine. In addition, using an enzyme in food manufacturing can also be one of such food ingredient conversion techniques.

It is known that, as a method for converting an organic compound using an enzyme, glucose can be oxidized (converted) with glucose dehydrogenase and nicotinamide adenine dinucleotide. For example, Japanese Unexamined Patent Application Publication No. 4-370755 discloses a glucose biosensor including a working electrode formed such that glucose dehydrogenase and nicotinamide adenine dinucleotide are adsorbed and immobilized on a particular electrode surface, although this is not a food ingredient conversion technique.

Incidentally, in an enzymatic reaction, there is a pH condition suitable for a particular reaction (also referred to as an optimum pH) for each type of enzyme. Japanese Unexamined Patent Application Publication No. 2-138862 discloses a measurement method using an enzyme electrode on which a hydrogen peroxide-generating oxidase is immobilized, in which the pH of a sample containing catalase is adjusted to 4 or less, an optimum pH in this enzymatic reaction is maintained, and measurement of the sample is performed with the enzyme electrode, although this is not a food ingredient conversion technique.

### SUMMARY

The methods for converting an organic compound (hereinafter, also referred to as an organic substance) using an enzyme or the configurations of devices therefor, etc. in the related art are not intended for food ingredient conversion; therefore, there is room for improvement in addressing the methods and configurations suitable for conversion of food ingredients.

As an example thereof, there is a known biosensor that involves the conversion of an organic compound using an enzyme to measure a required numerical value, such as quantification of a specific component in a system. In the case where the purpose is food ingredient conversion, it is necessary to convert a target organic compound in a large amount at low cost compared with such a biosensor, and there is room for improvement in performing more efficient conversion of the organic compound.

One non-limiting and exemplary embodiment provides a food ingredient conversion method that can achieve more efficient conversion of an organic compound in terms of pH condition of the reaction system. Another non-limiting and exemplary embodiment provides a food ingredient conversion device that can achieve more efficient conversion of an organic compound in terms of pH condition of the reaction system.

In one general aspect, the techniques disclosed here feature a food ingredient conversion method for converting an organic compound in a reaction system including an external liquid and contained in a reaction vessel, the reaction vessel having therein a working electrode that activates at least one of an enzyme or a coenzyme and a counter electrode, the food ingredient conversion method including activating at least one of the enzyme or the coenzyme by applying a voltage from an external power supply located outside the reaction vessel and connected to the working electrode and the counter electrode to cause a current to flow between the working electrode and the counter electrode; transferring a proton between the organic compound and the external liquid by an enzymatic reaction using at least one of the activated enzyme or coenzyme; acquiring information on the current that has been caused to flow by the external power supply; and determining, based on the acquired information on the current, an amount of a pH adjusting agent to be fed for adjusting a pH of the reaction system.

According to the food ingredient conversion method and the food ingredient conversion device according to aspects of the present disclosure, more efficient conversion of an organic compound can be performed in terms of pH condition of the reaction system.

It should be noted that general or specific embodiments may be implemented as a system, a method, an integrated circuit, a computer program, a storage medium, or any selective combination thereof.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1 is a view illustrating an example of the configuration of a food ingredient conversion device according to an embodiment;

FIG. 2 is a sectional view taken along line II-II in FIG. 1;

FIG. 3 is a schematic diagram for illustrating food ingredient conversion according to the embodiment;

FIG. 4 is a schematic diagram for illustrating food ingredient conversion according to Example 1 of the embodiment;

FIG. 5 is a flowchart illustrating a food ingredient conversion method according to Example 1 of the embodiment; and

FIG. 6 is a schematic diagram for illustrating food ingredient conversion according to Example 2 of the embodiment.

### DETAILED DESCRIPTIONS

### Underlying Knowledge Forming Basis of the Present Disclosure

Glucose dehydrogenase (GDH) functions as a catalyst in a D-glucose degradation reaction represented by a formula (1) below in which nicotinamide adenine dinucleotide (NADH) or nicotinamide adenine dinucleotide phosphate (NADPH) is involved as a coenzyme.

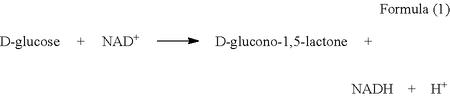

The reaction represented by the formula (1) is a reaction in which a proton (H⁺) is released (transferred) from D-glucose, which is an organic compound serving as a food ingredient, by an enzymatic reaction in the presence of an enzyme to convert the D-glucose into D-glucono-1,5-lactone. In this reaction, protons are released into the reaction system with the progress of the reaction, and thus the pH decreases (tends to become acidic). In that case, unless the pH of the reaction system is adjusted, with the progress of the reaction, the pH condition of the reaction system gradually deviates from the optimum pH (around pH 7.5 to 8.0) of glucose dehydrogenase, resulting in a decrease in reaction efficiency. Note that, in the formula (1), the proton balance does not match between the right side and the left side strictly because protons are supplied from water molecules, etc. in the system. In the case of using NADPH instead of NADH, NAD⁺ in the formula is replaced by NADP⁺.

On the other hand, a peroxidase functions as a catalyst in a degradation reaction of an organic compound represented by a formula (2) below through an enzymatic reaction.

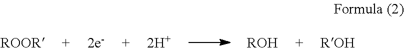

The reaction represented by the formula (2) is a reaction in which electrons (e⁻) are donated from an electron donor to an organic compound (represented by ROOR′ as a general formula), and protons are transferred from the outside of the organic compound (for example, water molecules in the system) to the organic compound to convert the organic compound to ROH and R′OH. In this reaction, protons in the reaction system are consumed with the progress of the reaction, and thus the pH increases (tends to become basic). In that case, unless the pH of the reaction system is adjusted, with the progress of the reaction, the pH condition of the reaction system gradually deviates from the optimum pH of the peroxidase, also resulting in a decrease in reaction efficiency.

The knowledge based on the formula (1) will be described below.

When the concentration of glucose in a predetermined sample is measured using the reaction represented by the formula (1), for example, the concentration of NADH generated per unit time is measured. In this case, the amount of NAD⁺ consumed for measuring the concentration of glucose is not so large. On the other hand, when glucose in a liquid containing glucose is degraded using the above-described reaction, it may be difficult to add NAD⁺ at a concentration commensurate with the amount of glucose contained in the liquid. This is because the concentration of NAD⁺ dissolved in the liquid has an upper limit (saturated concentration). In particular, in the case of using an enzymatic reaction, since solution conditions such as the optimum temperature and the optimum pH of the enzyme exist, it is often practically impossible to sufficiently increase the concentration of NAD⁺ in accordance with the amount of glucose.

Therefore, in the above reaction, one conceivable method is to oxidize generated NADH (that is inactive in terms of enzymatic reaction) to generate NAD⁺ that is activated again, thereby degrading glucose contained in a high concentration in a liquid even with the addition of a small amount of NAD⁺. For this purpose, glucose is sequentially degraded while NADH generated in the above-described reaction is continuously oxidized by an electrochemical method and the concentration of NAD⁺ in the liquid is maintained so as not to decrease, and thus glucose is degraded with high efficiency and continuously, while the cost in terms of energy for oxidizing NADH is kept low. The same applies to an example of the formula (2) in that a small amount of inactivated enzyme is activated by electrochemical assistance and continuously utilized. Specifically, the enzymatic reactions are continued while the reactions represented by the formulas (1) and (2) are assisted by an electrochemical method, and thus, protons can be added or released (transferred in either case) to an organic compound present in an amount relatively larger than an enzyme and/or a coenzyme even with a small amount of enzyme and/or coenzyme. In addition, these reactions can be performed at high energy efficiency.

In such a case, a small amount of NAD⁺ is repeatedly provided for a reaction; therefore, with the repeated reaction, the pH of the reaction system deviates from the optimum pH of the enzyme, and the reaction efficiency gradually decreases. In this case, if the pH of the reaction system can be adjusted by feeding a pH adjusting agent, the decrease in the reaction efficiency can be suppressed. However, in terms of food raw materials, for example, in a case where highly viscous ingredients such as monosaccharides, disaccharides, or polysaccharides are contained, or an alcohol is contained as a raw material, the measurement itself may be difficult with a typical pH meter, or the measured pH value may be deviated in some situations. In such a case, it is also conceivable that a pH adjusting agent is fed on the basis of, for example, the elapsed time of the reaction without measuring the pH of the reaction system (without an indicator of the pH); however, an enzymatic reaction is a sensitive reaction, and the way the pH changes may change depending on a slight difference in conditions. Accordingly, if a pH adjusting agent is fed without an indicator of the pH, excess or deficiency of the pH adjusting agent may occur, and the deviation from the optimum pH of the enzyme may be accelerated instead in some cases.

In view of the above, as a result of extensive studies, the present inventors have found that the transfer of electrons and the generation or consumption of protons due to a reaction occur simultaneously, and thus there is a correlation between the amount of inactivated enzyme or coenzyme and the amount of protons that has changed in the reaction system, in particular, in the external liquid, and there is a correlation also between the amount of inactivated enzyme or coenzyme and the current (amount of electrons) flowing from an electrode in order to activate the enzyme or coenzyme again, and that the amount of protons that has changed in the reaction system, in particular, in the external liquid can be estimated from information on the current that has been caused to flow from an external power supply. More specifically, if the information on the current that has been caused to flow from an external power supply is used as an indirect indicator of the pH, and a pH adjusting agent is fed on the basis of this information, the pH adjusting agent can be fed in an amount appropriate for the reaction. Consequently, the pH of the reaction system, in particular, of the external liquid can be appropriately maintained, and the conversion of an organic compound can be more efficiently performed in terms of pH condition of the reaction system. Furthermore, even in a reaction system in which a pH meter can be used, the pH of the external liquid can be appropriately maintained without using a pH meter, which is also advantageous in terms of cost.

### Aspects of Present Disclosure

The summary of aspects of the present disclosure is as follows.

According to a first aspect of the present disclosure, there is provided a food ingredient conversion method for converting an organic compound in a reaction system including an external liquid and contained in a reaction vessel, the reaction vessel having therein a working electrode that activates at least one of an enzyme or a coenzyme and a counter electrode, the food ingredient conversion method including activating at least one of the enzyme or the coenzyme by applying a voltage from an external power supply located outside the reaction vessel and connected to the working electrode and the counter electrode to cause a current to flow between the working electrode and the counter electrode, transferring a proton between the organic compound and the external liquid by an enzymatic reaction using at least one of the activated enzyme or coenzyme, acquiring information on the current that has been caused to flow by the external power supply, and determining, based on the acquired information on the current, an amount of a pH adjusting agent to be fed for adjusting a pH of the reaction system that has changed due to the transfer of the proton.

According to this food ingredient conversion method, the conversion of an organic compound can be performed by an enzymatic reaction using at least one of an activated enzyme or coenzyme. In this conversion, a reaction of a proton transfer in which a proton is added from the reaction system to the organic compound or a proton is eliminated from the organic compound to the reaction system is performed, and the amount of protons in the reaction system changes before and after the enzymatic reaction. Accordingly, the pH of the reaction system changes before and after the enzymatic reaction. On the other hand, at least one of the activated enzyme or coenzyme is inactivated during the reaction. In this food ingredient conversion method, the at least one of inactivated enzyme or coenzyme can be activated again by applying a voltage between a working electrode and a counter electrode to cause a current to flow between the working electrode and the counter electrode, and can be used for the enzymatic reaction. The total amount of current that has been caused to flow between the working electrode and the counter electrode in order to activate the at least one of inactivated enzyme or coenzyme correlates with the total amount of protons that has changed in the reaction system during the reaction. Therefore, the total amount of protons that has changed in the reaction system, that is, the pH that has changed can be estimated from the information on the current that has been caused to flow between the working electrode and the counter electrode. Accordingly, it is not necessary to measure the pH of the reaction system directly with a pH meter or the like, which is advantageous in terms of cost. Furthermore, for example, even in a situation in which the measurement itself is difficult with a typical pH meter, or the measured pH value deviates, a pH adjusting agent can be appropriately fed according to the estimated pH; therefore, for a greater variety of reaction systems, the conversion of a food ingredient that requires pH adjustment can be performed. Thus, according to the food ingredient conversion method, more efficient conversion of an organic compound can be performed in terms of pH condition of the reaction system.

According to a second aspect of the present disclosure, there is provided the food ingredient conversion method of the first aspect, further including adjusting the pH of the reaction system by feeding the determined amount of the pH adjusting agent to be fed.

According to this food ingredient conversion method, the pH adjusting agent can be appropriately fed in accordance with the estimated pH, and more efficient conversion of an organic compound can be performed in terms of pH condition of the reaction system.

According to a third aspect of the present disclosure, there is provided the food ingredient conversion method of the first or second aspect, wherein, in the transferring, the proton is transferred from the organic compound to the external liquid.

According to this food ingredient conversion method, the conversion of the organic compound in which a proton is transferred from the organic compound to the external liquid can be more efficiently performed in terms of pH condition of the reaction system.

According to a fourth aspect of the present disclosure, there is provided the food ingredient conversion method of the first or second aspect, wherein, in the transferring, the proton is transferred from the external liquid to the organic compound.

According to this food ingredient conversion method, the conversion of the organic compound in which a proton is transferred from the external liquid to the organic compound can be more efficiently performed in terms of pH condition of the reaction system.

According to a fifth aspect of the present disclosure, there is provided the food ingredient conversion method of any one of the first to fourth aspects, wherein the coenzyme is a redox coenzyme, an electron mediator that transfers an electron between the working electrode and the redox coenzyme is immobilized on the working electrode, and in the activating, an electron is transferred from the working electrode to the electron mediator, and at least one of the enzyme or the redox coenzyme is activated by the electron mediator to which the electron has been transferred.

According to this food ingredient conversion method, an action such as electron transfer easily occurs between the electron mediator and the working electrode, and thus the reaction efficiency can be increased.

According to a sixth aspect of the present disclosure, there is provided the food ingredient conversion method of the fifth aspect, wherein the redox coenzyme is NADH or NADPH.

According to this food ingredient conversion method, NADH or NADPH can be used as the redox coenzyme.

According to a seventh aspect of the present disclosure, there is provided the food ingredient conversion method of any one of the first to sixth aspects, wherein the organic compound includes at least one of a monosaccharide, a disaccharide, or a polysaccharide.

According to this food ingredient conversion method, an organic compound including at least one of a monosaccharide, a disaccharide, or a polysaccharide can be more efficiently converted in terms of pH condition of the reaction system.

According to an eighth aspect of the present disclosure, there is provided the food ingredient conversion method of any one of the first to sixth aspects, wherein the organic compound includes an alcohol.

According to this food ingredient conversion method, an organic compound including an alcohol can be more efficiently converted in terms of pH condition of the reaction system.

According to a ninth aspect of the present disclosure, there is provided the food ingredient conversion method of any one of the first to sixth aspects, wherein the organic compound has a disulfide bond.

According to this food ingredient conversion method, an organic compound having a disulfide bond can be more efficiently converted in terms of pH condition of the reaction system.

According to a tenth aspect of the present disclosure, there is provided the food ingredient conversion method of any one of the first to ninth aspects, wherein at least one of the enzyme or the coenzyme is immobilized on the working electrode.

According to this food ingredient conversion method, an action such as electron transfer easily occurs between at least one of the immobilized enzyme or coenzyme and the working electrode, and thus the reaction efficiency can be increased.

According to an eleventh aspect of the present disclosure, there is provided a food ingredient conversion device for converting an organic compound in a reaction system including an external liquid, the food ingredient conversion device including a working electrode that activates at least one of an enzyme or a coenzyme, a counter electrode, a reaction vessel that contains the reaction system, an external power supply that applies a voltage between the working electrode and the counter electrode to cause a current to flow between the working electrode and the counter electrode, an acquirer that acquires information on the current that has been caused to flow by the external power supply, and a determiner that determines, based on the acquired information on the current, an amount of a pH adjusting agent to be fed for adjusting a pH of the reaction system that has changed due to a proton transfer caused by application of the current.

According to this food ingredient conversion device, advantageous effects similar to those of the food ingredient conversion method can be produced.

According to a twelfth aspect of the present disclosure, there is provided the food ingredient conversion device of the eleventh aspect, wherein the enzyme is an oxidase.

According to this food ingredient conversion device, an organic compound can be efficiently converted by an enzymatic reaction in the presence of an oxidase.

According to a thirteenth aspect of the present disclosure, there is provided the food ingredient conversion device of the eleventh aspect, wherein the enzyme is a reductase.

According to this food ingredient conversion device, an organic compound can be efficiently converted by an enzymatic reaction in the presence of a reductase.

According to a fourteenth aspect of the present disclosure, there is provided the food ingredient conversion device of any one of the eleventh to thirteenth aspects, wherein the enzyme and the coenzyme are immobilized on the working electrode.

According to this food ingredient conversion device, an action such as electron transfer easily occurs between the enzyme and coenzyme and the working electrode, and thus the reaction efficiency can be increased.

It should be noted that these general or specific aspects may be implemented as a system, a method, a device, an integrated circuit, a computer program, or a computer-readable recording medium such as a CD-ROM or may be implemented as a combination of any of a system, a method, a device, an integrated circuit, a computer program, and a recording medium.

Hereinafter, embodiments will be specifically described with reference to the drawings.

It should be noted that all of the embodiments described below are general or specific examples. Numerical values, shapes, materials, constituent elements, the arrangement positions and connection forms of constituent elements, steps, the order of steps, etc. described in the following embodiments are illustrative and are not intended to limit the scope of the claims. Of the constituent elements described in the following embodiments, those which are not recited in independent claims representing the broadest concepts are described as optional constituent elements. Furthermore, each drawing is not necessarily a precise drawing. In the drawings, configurations that are substantially identical are assigned the same reference signs, and a duplicated description thereof may be omitted or simplified.

In the following description of the drawings, the X-axis direction, the Y-axis direction, and the Z-axis direction that are orthogonal to each other will be used as appropriate. In particular, in the Z-axis direction, the positive side may be referred to as an upper side, and the negative side may be referred to as a lower side in the following description.

In the present disclosure, terms that indicate relationships between elements, such as parallel and perpendicular, terms that indicate the forms of elements, such as rectangular, and numerical values not only have their precise meanings but also have meanings that include substantially the same ranges, for example, differences of about several percent.

In the drawings of the present disclosure, broken lines indicate objects that are not visible from the surface, and boundaries between regions.

### Embodiments

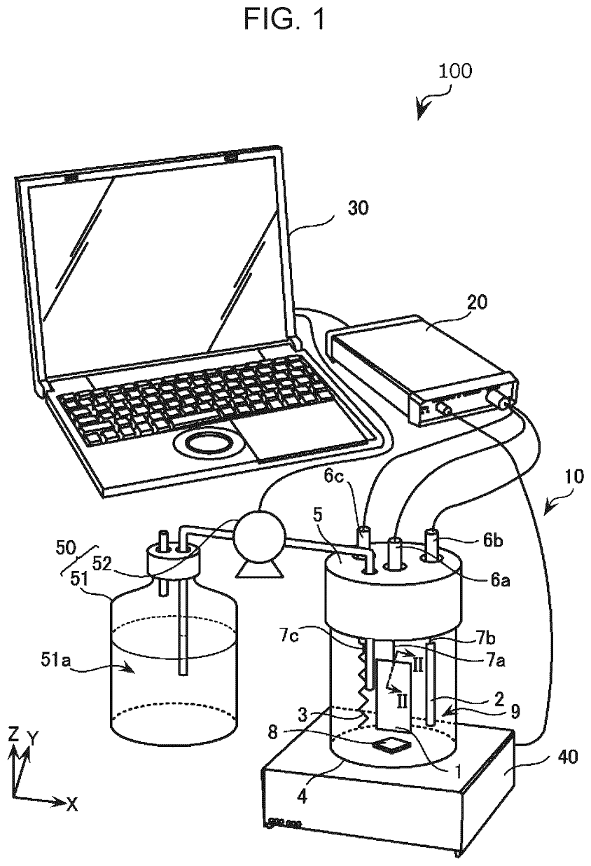

Embodiments will be specifically described below with reference to FIGS. 1 to 6.

### Food Ingredient Conversion Device

### 1. Overview

The overview of a food ingredient conversion device (hereinafter, may be simply referred to as a device) according to an embodiment will be first described with reference to FIG. 1. FIG. 1 is a view illustrating an example of the configuration of a food ingredient conversion device according to the present embodiment.

A food ingredient conversion device 100 performs electron transport between an electrode and an enzyme and/or a coenzyme upon application of a voltage to the electrode. For example, the food ingredient conversion device 100 donates electrons from an electrode to an enzyme immobilized on the surface of the electrode. This causes electron transfer between the electrode and a molecule of an organic compound (hereinafter, also referred to as a target molecule) in a sample, and the target molecule is oxidized or reduced. For example, in the food ingredient conversion device 100, a voltage is applied to the electrode in a state where a sample (which is one example of the reaction system and is also referred to as, for example, a sample solution) containing an external liquid and target molecules is in a non-flowing state, electron transfer is thereby performed between an enzyme and/or a coenzyme immobilized on the electrode and the target molecules in the sample, and the target molecules are reduced. Subsequently, the liquid is changed from the non-flowing state to a flowing state, thereby diffusing the reduced target molecules into the liquid. By repeating the change in the flowing state of the liquid in this manner, the target molecules can be efficiently reduced throughout the liquid.

The external liquid is a main component forming the reaction system and is composed of a fluid such as water or an alcohol serving as a main component and various additives. The external liquid may have, in addition to a function of stabilizing, for example, the orientation of hydrophilic groups in the reaction system with a hydration bond or the like, a buffer function of suppressing an increase (or decrease) in pH when protons or the like are generated and the pH increases (or protons are consumed and the pH decreases) in the reaction system. Therefore, a buffer component composed of an acid or a base and a salt may be dissolved in the external liquid.

In this case, during the reaction, the transfer of protons occurs between target molecules and the external liquid. In the case where protons are released from target molecules, protons preferably leave the vicinity of the generated substance so as to prevent the released protons from being donated again to the substance generated after the reaction and causing the reverse reaction. On the other hand, in the case where protons are donated to target molecules, preferably, protons are rapidly supplied to the vicinity of the target substance. For this purpose, it is effective to perform stirring as described above. The expression “a liquid is in a non-flowing state” refers to, for example, a state in which the liquid is not stirred or shaken (that is, is not subjected to any external force such as shear force or vibration), and no movement such as shaking is observed on the liquid surface.

### 2. Configuration

Next, the configuration of a food ingredient conversion device 100 according to an embodiment will be described with reference to FIGS. 1 to 3.

As illustrated in FIG. 1, a food ingredient conversion device 100 includes a stirring unit 40 that stirs a sample 9 containing target molecules (here, an organic compound before conversion) and an external liquid to cause the sample to be in a flowing state, an electrode that performs electron transfer with the target molecules to transfer protons between the target molecules and the external liquid, an external power supply 20 that applies a voltage to the electrode, a pH adjusting unit 50 that feeds a pH adjusting agent 51a from the outside to the sample 9 to adjust the pH of the sample 9, and a controller 30 that controls the external power supply 20, the stirring unit 40, and the pH adjusting unit 50.

A voltage application unit 10 is, for example, a three-electrode cell including an electrode 1 (also referred to as a working electrode), a reference electrode 2, a counter electrode 3, a cell 4, a lid 5, terminals 6a, 6b, and 6c, and leads 7a, 7b, and 7c. Alternatively, the voltage application unit 10 may be, for example, a two-electrode cell including a working electrode (electrode 1) and a counter electrode 3.

The electrode 1 and the counter electrode 3 are composed of an electrically conductive substance. The electrically conductive substance may be, for example, a carbon material, a conductive polymer material, a semiconductor, or a metal.

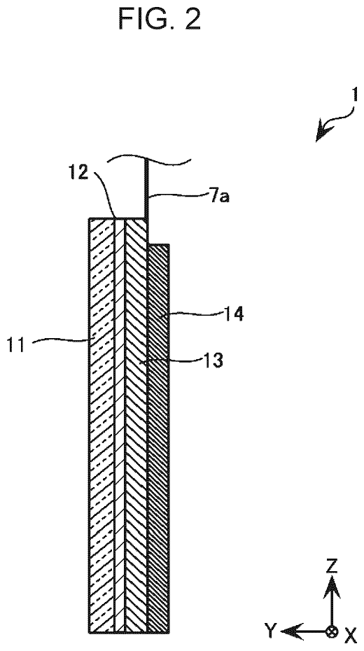

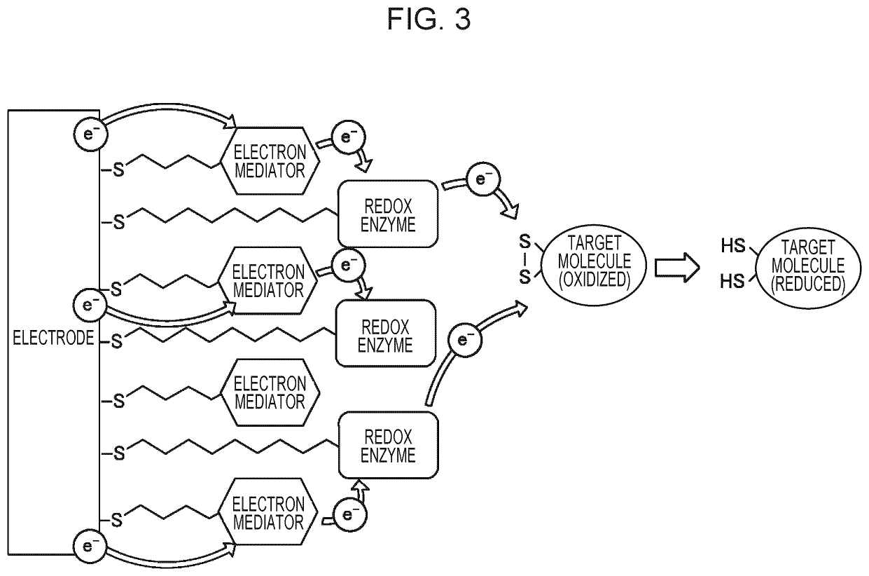

First, the electrode 1 will be described with reference to FIGS. 2 and 3. FIG. 2 is a sectional view taken along line II-II in FIG. 1. FIG. 3 is a schematic diagram illustrating an enzyme and/or a coenzyme immobilized on an electrode, etc.

The electrode 1 is an electrode on which an enzyme and/or a coenzyme is immobilized. The electrode 1 includes, for example, a glass substrate 11, a titanium vapor-deposited layer 12 formed on the glass substrate 11 by vapor deposition, a substrate 13 formed on the titanium vapor-deposited layer 12, and a reaction layer 14 including an enzyme and/or a coenzyme immobilized on the substrate 13. An electrode on which an enzyme, a coenzyme, and an electron mediator (described later) are immobilized will be described below; however, each of the enzyme, the coenzyme, and the electron mediator may be merely contained in a flowable state as one component in the reaction system without being immobilized on the electrode. Alternatively, one or two of the enzyme, the coenzyme, and the electron mediator may be immobilized, and the other may be contained in a flowable state as one component in the reaction system.

The substrate 13 may be an electrically conductive substrate composed of, for example, gold, platinum, a carbon material such as glassy carbon, graphite, or boron-doped diamond, or tin-added indium oxide (ITO: indium tin oxide). The thickness of the substrate 13 is not particularly limited.

The shape of the electrode 1 is a plate shape formed of the substrate 13, but the shape of the electrode 1 is not limited to a specific shape. The electrode 1 may be linear, plate-like, or mesh-like or may be a fiber assembly. From the viewpoint of reaction efficiency, the electrode 1 is preferably designed so as to have a large surface area and have a cross-sectional area that provides low resistance.

An electron mediator involved in electron transfer is also immobilized on the substrate 13, and furthermore, a coenzyme may be immobilized. The electron mediator is a substance that performs electron transfer between the electrode and the enzyme immobilized on the electrode and may be, for example, viologen, a quinone, or indophenol.

The immobilization of the enzyme, coenzyme, and electron mediator suppresses the transfer of these molecular species from the vicinity of the electrode 1 and thus is effective from the viewpoint of reaction efficiency. In other words, the immobilization of the enzyme, coenzyme, and electron mediator is effective in view of mobility of molecular species. However, for some molecular species, immobilization may result in an orientation that is not suitable for reaction. Preferably, such molecular species are not immobilized in view of reactivity. That is, in the optimal configuration in this case, molecular species with no or negligible decrease in reactivity are immobilized, whereas molecular species with a decrease in reactivity are not immobilized.

The enzyme and/or coenzyme immobilized on the surface of the electrode (so-called substrate 13) is a protein that oxidizes or reduces a target molecule or a coenzyme that acts cooperatively with the protein. As illustrated in FIG. 3, the enzyme, coenzyme, and electron mediator are immobilized on the electrode (substrate 13) with a chain linker. The linker includes, for example, an alkyl chain having greater than or equal to 1 and less than or equal to 14 carbon atoms. Alternatively, the enzyme, coenzyme, and electron mediator may be immobilized directly on the surface of the electrode without a linker therebetween. Note that the phrase “without a linker therebetween” means that other molecular species are not interposed between the surface of the electrode and an enzyme, a coenzyme, or an electron mediator. However, a case where a portion of the main chain or a side chain constituting an enzyme, the portion being involved in immobilization, acts like a linker is also included in the case of “without a linker therebetween”.

The reference electrode 2 is an electrode that does not react with a component in the sample 9 and maintains a constant potential, and is used so that the potential difference between the electrode 1 and the reference electrode 2 is controlled to remain constant by the external power supply 20. The reference electrode 2 is, for example, a silver/silver chloride electrode. Note that the food ingredient conversion device 100 may include only the electrode 1 and the counter electrode 3 without including the reference electrode 2.

The counter electrode 3 is preferably, for example, a platinum electrode in view of the case of electron emission to maintain electrical neutrality. As the counter electrode 3, an electrically conductive substrate composed of, for example, gold, platinum, a carbon material such as glassy carbon, graphite, or boron-doped diamond, or tin-added indium oxide (ITO: indium tin oxide) may be used as in the electrode 1. The shape of the counter electrode 3 is also not limited to a specific shape. The counter electrode 3 may be linear, plate-like, or mesh-like or may be a fiber assembly. From the viewpoint of reaction efficiency, the counter electrode 3 is preferably designed so as to have a large surface area and have a cross-sectional area that provides low resistance.

In the present embodiment, the cell 4 contains the reaction system and is isolated from the outside. The cell 4 is an example of a reaction vessel that contains the reaction system and is a container in which the electrode 1, the reference electrode 2, and the counter electrode 3 are disposed.

The external power supply 20 applies a voltage between the electrode 1 and the counter electrode 3 of the voltage application unit 10 in accordance with a control signal output from the controller 30 and controls the potential between the electrode 1 and the reference electrode 2 to a predetermined value. As a result, a current corresponding to the potential between the electrode 1 and the reference electrode 2 flows between the electrode 1 and the counter electrode 3. The external power supply 20 transmits the control results (for example, the applied voltage, the value of the current that has flowed, the elapsed time during which the voltage has been applied, etc.) to the controller 30. This allows the controller 30 to grasp whether or not the control of the external power supply 20 is properly performed.

The controller 30 executes information processing for controlling the voltage application by the external power supply 20 and the movement of a motor (not illustrated) of the stirring unit 40, information processing for acquiring the above control results from the external power supply 20, information processing for determining an amount of pH adjusting agent 51a to be fed for adjusting the pH of the reaction system, and information processing for controlling the movement of the pH adjusting unit 50. Accordingly, the controller 30 functions as an example of an acquirer and a determiner. The controller 30 is realized by, for example, a processor, a microcomputer, or a dedicated circuit.

The stirring unit 40 controls the operation of the motor in accordance with a control signal output from the controller 30 to thereby control the rotation speed and the rotation time of a stirring element 8 set in the cell 4.

The pH adjusting unit 50 includes a tank 51 that contains a fluid for pH adjustment serving as the pH adjusting agent 51a and a pump 52 that actively feeds the fluid contained in the tank 51 into the cell 4. In the pH adjusting unit 50, the pump operates in accordance with an amount of pH adjusting agent 51a to be fed, the amount being determined by the controller 30, thereby feeding the pH adjusting agent 51a into the cell 4 in the determined amount to be fed. The drawing illustrates, as the pH adjusting agent 51a, a liquid substance of a base such as NaOH or an acid such as HCl. The pH adjusting agent 51a is preferably a substance that finally becomes edible through treatment such as neutralization so that no inconvenience occurs when it is added to food. The pump 52 is configured to suction the liquid in the tank 51 and supply the liquid to the cell 4. In this case, air can be supplied from the outside to the tank 51 so that the pressure in the tank 51 does not become negative. Alternatively, the pH adjusting unit 50 may be configured so that the pump 52 pressurizes the tank 51 to push out the liquid and supply the liquid to the cell 4.

Alternatively, a gaseous substance can also be used as the pH adjusting agent 51a. For example, CO₂ gas is a gaseous pH adjusting agent 51a that is advantageous in terms of being edible. In this case, the tank 51 is filled with high-pressure CO₂ gas (or liquefied CO₂) in advance, and an electromagnetic valve or the like is used instead of the pump 52, thereby feeding the CO₂ gas into the cell 4 by a gas pressure. In the case where the fluid is a gas, by disposing the outlet of a flow path of the pH adjusting agent 51a (path connecting the tank 51 and the cell 4 to each other) at a position lower than the liquid level, the gas is bubbled in the liquid in the reaction system, and thus the gas is easily dissolved. The pH adjusting unit 50 transmits the control results (for example, the amount of liquid that has been fed by the pump 52, the remaining amount of the pH adjusting agent 51a, etc.) to the controller 30. This allows the controller 30 to grasp whether or not the control of the pH adjusting unit 50 is properly performed.

In a method for producing an electrode on which an enzyme, a coenzyme, and an electron mediator are immobilized, first, a linker for immobilizing an enzyme, a coenzyme, and an electron mediator is immobilized on an electrode. The linker is dissolved in a solution of ethanol:acetonitrile=1:1, and the electrode is immersed in the resulting solution and allowed to stand for greater than or equal to one hour to immobilize the linker. The linker is preferably an alkyl chain having less than or equal to 14 carbon atoms so that electrons can transfer from the electrode to the enzyme, the coenzyme, and the electron mediator. For example, the linker is an alkyl chain having greater than or equal to 1 and less than or equal to 14 carbon atoms. In particular, the linker has, at one end thereof, a functional group capable of forming a bond with the enzyme, coenzyme, and electron mediator, such as a carboxyl group or an amino group and has, at the other end thereof, a thiol group.

Subsequently, the linker having, for example, a thiol group immobilized on the electrode through the above-described operation is bonded to the enzyme, coenzyme, and electron mediator. At this time, for example, in a case of an enzyme, an amino group at the N-terminal or in a side chain and a carboxyl group of the linker are bonded together by an amine coupling reaction. Thus, the enzyme is immobilized on the electrode with the linker therebetween. The coenzyme and the electron mediator are also immobilized on the electrode with a linker therebetween by selecting an appropriate functional group. When the enzyme, coenzyme, and electron mediator are immobilized directly on the electrode, the step of immobilizing the linker is omitted. The operations described above are merely examples. To immobilize the enzyme, coenzyme, and electron mediator on the electrode, it is possible to apply any existing technique, such as a technique of using an acid electroconductive polymer, a crosslinking agent, or the like or a technique of achieving immobilization with a monomolecular film-forming molecule therebetween.

### 3-1. Example 1

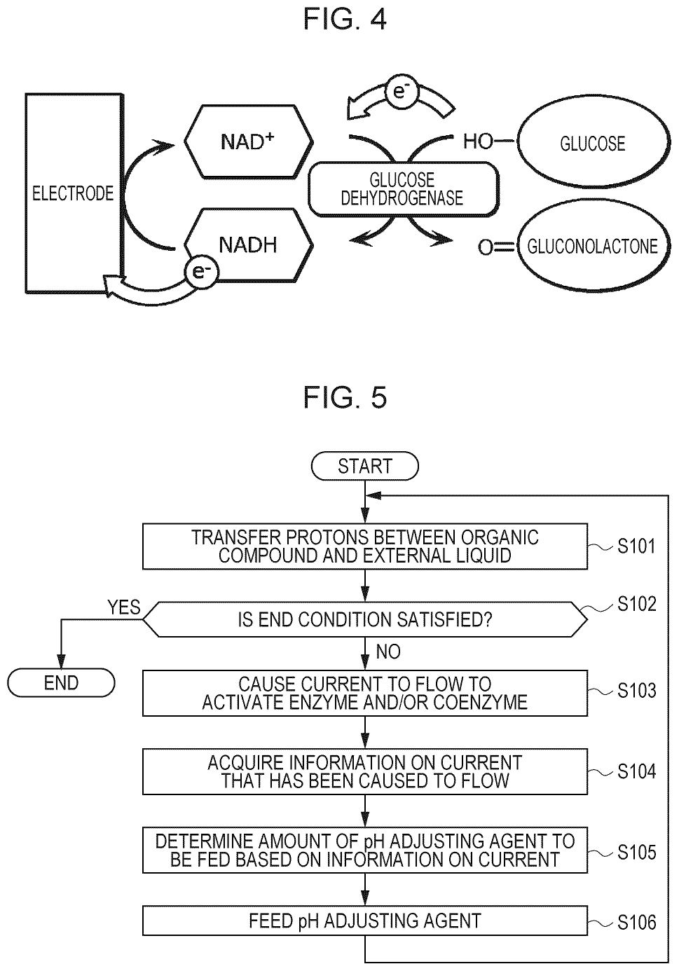

Hereinafter, an example of the degradation action of glucose by a dehydrogenase will be described as Example 1 with reference to FIGS. 4 and 5. FIG. 4 is a schematic diagram for illustrating food ingredient conversion according to Example 1 of the present embodiment. FIG. 5 is a flowchart illustrating a food ingredient conversion method according to Example 1 of the present embodiment.

In Example 1, the degradation of glucose using the reaction represented by the formula (1) is performed. Here, NAD⁺ and glucose dehydrogenase act cooperatively to cause a reaction in which a proton is released from glucose to generate gluconolactone. Accordingly, NAD⁺ is reduced and turned to NADH. In the reaction of this example, the transfer of protons between the organic compound (glucose) and the external liquid means the transfer of protons from the organic compound to the external liquid. Specifically, a proton transfers from glucose to NAD⁺ to generate NADH, NADH is subsequently reduced, and the proton thereby transfers to the external liquid (reaction step S101). Therefore, protons accumulate in the cell 4, and the pH decreases. A step S102 will be described later. In order to reduce NADH to NAD⁺ again as described above, the external power supply 20 applies a voltage between the electrode 1 and the counter electrode 3 so that the electrode 1 serves as a positive electrode and the counter electrode 3 serves as a negative electrode. A current flows in the reaction system between the electrode 1 and the counter electrode 3. At that time, near the electrode 1, an electron is released from NADH and is attracted to the electrode 1 to generate NAD⁺ (activation of coenzyme), and thus glucose dehydrogenase is again in a state of being capable of functioning as a catalyst (activation step S103). At this time, a proton is released from NADH to the external liquid. Repeating the reaction step S101 and the activation step S103 enables a large amount of glucose to be sequentially degraded even with a small amount of enzyme and/or coenzyme to perform appropriate conversion of the organic compound in terms of efficiency. While the reaction step S101 and the activation step S103 are repeated, a determination as to whether or not an end condition is satisfied (step S102) is performed, and if the end condition is satisfied (Yes in S102), the reaction is terminated. On the other hand, if the end condition is not satisfied (No in S102), the reaction step S101 and the activation step S103 are repeatedly performed. The end condition is that, for example, the amount of generated organic compound (gluconolactone in this example) reaches a certain level or more or a certain period of time has elapsed since the start of the reaction. Here, the step S102 is described as being performed between the reaction step S101 and the activation step S103. Alternatively, the step S102 may be performed before the reaction step S101 or may be performed after the activation step S103.

After the activation step S103, the controller 30 acquires information on the current that has been caused to flow from the external power supply 20 (acquisition step S104). For example, the controller 30 acquires the control results, thus performing the acquisition step S104. The information on the current that has been caused to flow is, for example, an integrated value of the current value in the time domain. The magnitude of the current value corresponds to the amount of activated enzyme and/or coenzyme at the moment, and thus can be read as the amount of protons released at the moment. Accordingly, the integrated value of the current value in the time domain corresponds to the amount of protons accumulated over time. Note that the information on the current that has been caused to flow may be acquired as an instantaneous value of the current by the controller, and the integrated value in the time domain may be calculated in the controller 30. That is, it is only necessary that the information on the current include at least information on the instantaneous value of the current.

The controller 30 replaces the information on the current that has been caused to flow, the information being acquired as described above, with an amount of accumulated protons, and determines, on the basis of the amount, an amount of pH adjusting agent 51a to be fed (determination step S105). The pH adjusting agent 51a used here is a basic fluid. The controller 30 determines the amount of pH adjusting agent 51a to be fed, for example, with reference to a database showing the relationship between the information on the current that has been caused to flow and the amount of pH adjusting agent 51a to be fed. Specifically, the database shows the relationship between the integrated value of the current value in the time domain and a total feed amount of pH adjusting agent 51a, and an amount corresponding to the difference between a total feed amount of pH adjusting agent 51a to be fed based on the integrated value of the current value in the time domain at that time and a feed amount of pH adjusting agent 51a that has been fed at that time is determined as the amount to be fed. The controller 30 generates a control signal that controls the pH adjusting unit 50 in accordance with the determined amount of pH adjusting agent 51a to be fed and outputs the control signal to the pH adjusting unit 50. Accordingly, the pH adjusting unit 50 feeds the pH adjusting agent 51a in the determined amount of pH adjusting agent 51a to be fed (pH adjusting step S106). As a result, the information on the current that has been caused to flow is used as an indirect indicator of pH, and the pH adjusting agent 51a can be fed on the basis of this indicator; therefore, the pH adjusting agent 51a can be fed in a feed amount appropriate for the reaction. Consequently, the pH of the reaction system, in particular, of the external liquid can be appropriately maintained, and the conversion of the organic compound can be more efficiently performed in terms of pH condition of the reaction system.

The reaction step S101, the step S102, and the activation step S103 (hereinafter, referred to as steps related to the external power supply 20), and the acquisition step S104, the determination step S105, and the pH adjusting step S106 (hereinafter, referred to as steps related to the pH adjusting unit 50) are each independently repeatedly performed. Accordingly, as illustrated in FIG. 5, the steps related to the pH adjusting unit 50 may be performed once for the steps related to the external power supply 20 performed once, or the steps related to the pH adjusting unit 50 may be performed once for the steps related to the external power supply 20 performed several times.

The frequency of the steps related to the pH adjusting unit 50 relative to the steps related to the external power supply 20 may depend on the time resolution of the operation of the pH adjusting unit 50, may depend on the time required for the fed pH adjusting agent 51a to be sufficiently stirred, or may be any frequency set by the user.

### 3-2. Example 2

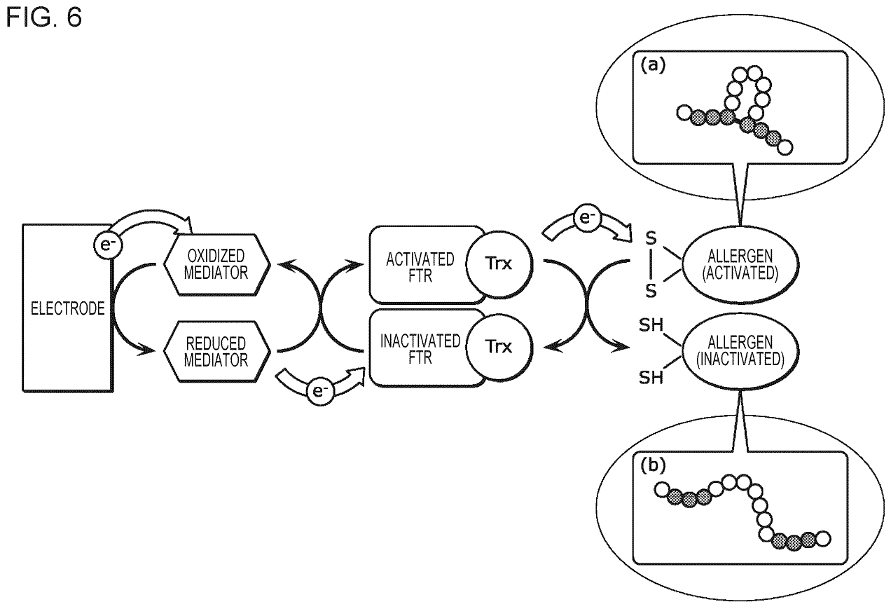

Hereinafter, an example of the reduction action of an allergen by a reductase will be described as Example 2 with reference to FIG. 6 together with FIG. 5. FIG. 6 is a schematic diagram for illustrating food ingredient conversion according to Example 2 of the present embodiment.

In Example 2, the reduction of a protein having a disulfide bond (—S—S—) is performed. Here, electrons are transferred sequentially through an electron mediator and a ferredoxin-thioredoxin reductase (FTR)-thioredoxin complex to cause a reaction in which the disulfide bond of the protein is reduced and cleaved. Accordingly, the electron mediator and the FTR-thioredoxin complex are reduced and inactivated. On the other hand, protons in the external liquid are consumed to reduce the disulfide bond to two thiol groups (—SH). In the reaction of this example, the transfer of protons between the organic compound (protein) and the external liquid means the transfer of protons from the external liquid to the organic compound. Specifically, protons in the external liquid are consumed to cause the above reaction in which the disulfide bond is reduced and cleaved, and the protons transfer from the external liquid to the protein (reaction step S101).

The external power supply 20 applies a voltage between the electrode 1 and the counter electrode 3 so that the electrode 1 serves as a negative electrode and the counter electrode 3 serves as a positive electrode. A current flows in the reaction system between the electrode 1 and the counter electrode 3. At that time, near the electrode 1, electrons are donated to the inactivated electron mediator to activate the electron mediator, and the activated electron mediator donates electrons to the inactivated FTR-thioredoxin complex to activate the FTR-thioredoxin complex, so that the FTR-thioredoxin complex is again in a state of being capable of reducing the protein (activation step S103). Repeating the reaction step S101 and the activation step S103 enables a large amount of protein to be sequentially reduced even with a small amount of enzyme and/or coenzyme to perform appropriate conversion of the organic compound in terms of efficiency. While the reaction step S101 and the activation step S103 are repeated, a determination as to whether or not an end condition is satisfied (step S102) is performed, and if the end condition is satisfied (Yes in S102), the reaction is terminated. On the other hand, if the end condition is not satisfied (No in S102), the reaction step S101 and the activation step S103 are repeatedly performed. The end condition is that, for example, the amount of generated organic compound (reduced protein having thiol groups in this example) reaches a certain level or more or a certain period of time has elapsed since the start of the reaction.

For the above steps related to the external power supply 20, the steps related to the pH adjusting unit 50 are performed at a predetermined frequency as in Example 1.

The protein in this example is assumed to be an allergen having a disulfide bond (—S—S—). Examples of allergenic proteins having disulfide bonds (hereinafter also simply referred to as allergenic proteins) include allergens derived from food such as beans, wheat, milk, seafood, eggs, and rice, environments such as pollen, animals, nematodes, filamentous fungi, molds, and ticks, and animals such as animal furs and dander. In particular, in the case of considering food, typical examples of the allergenic proteins include prolamins, ovalbumin, and β-lactoglobulin.

The disulfide bond in the allergen is reduced to thiol groups (—SH) as electrons are donated from an activated FTR. FIG. 6 schematically illustrates, above the allergen (activated), a portion (a) containing a disulfide bond of the allergen before reduction. FIG. 6 also schematically illustrates, below the allergen (inactivated), a portion (b) in the allergen after reduction corresponding to (a) above. Open circles illustrated in (a) and (b) each indicate one amino acid residue, and hatched circles each indicate an amino acid residue that the IgE antibody recognizes (epitope). As illustrated in (a) and (b), when the disulfide bond of the allergen is reduced, a loop portion of the amino acid sequence is loosened, and thus a different amino acid sequence is inserted in the middle of the amino acid sequence that the IgE antibody recognizes. This increases the possibility that the IgE antibody can no longer recognize the specific binding site. Thus, the allergenicity of the allergen is weakened.

Note that, although a loop portion has been described as an example in (a) and (b) above, the portion is not limited to this. In a three-dimensional structure of a protein, a functional site is formed as amino acid residues located far from each other in the amino acid sequence of the peptide chain of the protein come close to each other. Therefore, when the disulfide bond is reduced, the linkage between secondary structures of the protein that have been linked together through the disulfide bond breaks, and the functional site formed by the linkage between the secondary structures is no longer maintained. More specifically, a disulfide bond is a bond that links secondary structures of the protein to each other to further strengthen the three-dimensional structure of the protein. Accordingly, when the disulfide bond is cleaved, the linkage between the secondary structures breaks, and the flexibility (i.e., fluctuation) of the three-dimensional structure of the allergenic protein increases. As a result, the functional site of the allergenic protein (for example, a conformational epitope) is less likely to be maintained; therefore, this increases the possibility that the IgE antibody can no longer recognize the specific binding site of the allergenic protein.

Furthermore, as a result of an increase in the flexibility (i.e., fluctuation) of the three-dimensional structure of the allergenic protein due to the reduction of the disulfide bond, digestive enzymes are likely to act on cleavage sites of the peptide chain of the allergenic protein to be cleaved by the digestive enzymes; therefore, the allergenic protein is more easily digested by the digestive enzymes.

The description has been given above, and the organic compound treated by the food ingredient conversion device 100 may be an organic compound including not only glucose, which is a monosaccharide, but also disaccharides such as sucrose (cane sugar, sugar), lactose (milk sugar), and maltose (malt sugar) and polysaccharides such as starch, and is not limited to a specific raw material. For example, a trace amount of glucose contained in egg white, etc. can also be selectively degraded. Moreover, an appropriate enzyme, coenzyme, electron mediator, and the like suitable for the raw material to be converted can be selected. For example, when glucose is converted into gluconolactone, glucose dehydrogenase and NADH can be used as the enzyme and the coenzyme, respectively.

The organic compound treated by the food ingredient conversion device 100 may be an organic compound containing an alcohol, and the reaction formulas in such a case are as shown in formulas (3), (4), and (5) below. Here, a reaction (conversion) in which an alcohol dehydrogenase is used as an enzyme, and an aldehyde is generated using NAD⁺ to decrease an alcohol can be performed.

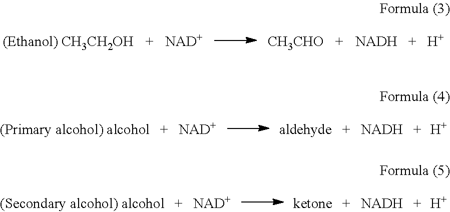

The food ingredient conversion method and the food ingredient conversion device according to the present disclosure have been described above on the basis of embodiments, but the present disclosure is not limited to these embodiments. Various modifications of the embodiments that are conceivable by those skilled in the art and other embodiments obtained by combining some of the constituent elements in the embodiments are also included in the scope of the present disclosure so long as they do not depart from the spirit of the present disclosure.

The food ingredient conversion method and the like according to the present disclosure are useful for improving the efficiency of food ingredient conversion.

What is claimed is:

1. A food ingredient conversion method for converting an organic compound in a reaction system including an external liquid and contained in a reaction vessel,
the reaction vessel having therein a working electrode that activates at least one of an enzyme or a coenzyme and a counter electrode,
the food ingredient conversion method comprising:
activating at least one of the enzyme or the coenzyme by applying a voltage from an external power supply located outside the reaction vessel and connected to the working electrode and the counter electrode to cause a current to flow between the working electrode and the counter electrode;
transferring a proton between the organic compound and the external liquid by an enzymatic reaction using at least one of the activated enzyme or coenzyme;
acquiring information on the current that has been caused to flow by the external power supply; and
determining, based on the acquired information on the current, an amount of a pH adjusting agent to be fed for adjusting a pH of the reaction system.

2. The food ingredient conversion method according to claim 1, further comprising:
adjusting the pH of the reaction system by feeding the determined amount of the pH adjusting agent to be fed.

3. The food ingredient conversion method according to claim 1,
wherein, in the transferring, the proton is transferred from the organic compound to the external liquid.

4. The food ingredient conversion method according to claim 1,
wherein, in the transferring, the proton is transferred from the external liquid to the organic compound.

5. The food ingredient conversion method according to claim 1,
wherein the coenzyme is a redox coenzyme,
an electron mediator that transfers an electron between the working electrode and the redox coenzyme is immobilized on the working electrode, and
in the activating,
an electron is transferred from the working electrode to the electron mediator, and
at least one of the enzyme or the redox coenzyme is activated by the electron mediator to which the electron has been transferred.

6. The food ingredient conversion method according to claim 5,
wherein the redox coenzyme is NADH or NADPH.

7. The food ingredient conversion method according to claim 1,
wherein the organic compound includes at least one of a monosaccharide, a disaccharide, or a polysaccharide.

8. The food ingredient conversion method according to claim 1,
wherein the organic compound includes an alcohol.

9. The food ingredient conversion method according to claim 1,
wherein the organic compound has a disulfide bond.

10. The food ingredient conversion method according to claim 1,
wherein at least one of the enzyme or the coenzyme is immobilized on the working electrode.

11. A food ingredient conversion device for converting an organic compound in a reaction system including an external liquid, the food ingredient conversion device comprising:
a working electrode that activates at least one of an enzyme or a coenzyme;
a counter electrode;
a reaction vessel that contains the reaction system;
an external power supply that applies a voltage between the working electrode and the counter electrode to cause a current to flow between the working electrode and the counter electrode;
an acquirer that acquires information on the current that has been caused to flow by the external power supply; and
a determiner that determines, based on the acquired information on the current, an amount of a pH adjusting agent to be fed for adjusting a pH of the reaction system.

12. The food ingredient conversion device according to claim 11,
wherein the enzyme is an oxidase.

13. The food ingredient conversion device according to claim 11,
wherein the enzyme is a reductase.

14. The food ingredient conversion device according to claim 11,
wherein the enzyme and the coenzyme are immobilized on the working electrode.
